(19) United States
(12) Patent Application Publication
(10) Pub. No.: US 2008/0132831 A1
(43) Pub. Date: Jun. 5, 2008

(54) Suppository insertion assembly

(76) Inventor: Charles Temple, Love Ladies, NJ (US)

Correspondence Address: CLIFFORD G. FRAYNE, 136 DRUM POINT RD, SUITE 7A, BRICK, NJ, 08723, US

(21) Appl. No.: 11/605,633

(22) Filed: Nov. 30, 2006

(51) Int. Cl. A61M 31/00

(52) U.S. Cl. 604/60; 604/288

(57) ABSTRACT

A suppository insertion assembly having a first end having a sealable reservoir member and associated plunger and piston, the reservoir member in first conduit communication with a disposable suppository retaining member, the suppository retaining member having a second conduit means snap fit and securable with the first conduit means of the reservoir member, the reservoir member for receipt of a petroleum jelly cartridge, or similar pressurized fluid, the plunger-piston member of the reservoir member compressing the petroleum jelly cartridge so as to cause petroleum jelly to pass through the first and second conduit means and to exert pressure on the suppository, thereby ejecting the suppository from the suppository retention member into the rectum.

### BACKGROUND OF THE INVENTION

1. Field of the Invention

The present invention relates to suppositories, and more particularly, to an insertion assembly to more easily insert a suppository.

2. Description of the Prior Art

Suppositories are used in a variety of gastrointestinal situations. The suppository is inserted in the rectum oftentimes to relieve constipation. In other situations, the suppository is prescribed prior to undergoing a gastro intestinal medical procedure, such as a colonoscopy and for application of medicines that cannot be taken orally.

The suppository may be applied by medical personnel, but most often is applied individually by the user. The manner of application by the individual user is oftentimes difficult, depending upon their physical situation, requiring them to position themselves in an uncomfortable position.

Applicant has developed an insertion assembly which more easily allows the individual or medical personnel to apply a suppository without having to assume an uncomfortable position.

### OBJECTS OF THE INVENTION

An object of the present invention is to provide for a novel suppository insertion assembly.

A further object of the present invention is to provide for a novel suppository insertion assembly which does not require the individual user to assume an uncomfortable or unnatural physical position.

A still further object of the present invention is to provide for a novel suppository insertion assembly in kit form which allows for the disposal of a portion of the assembly after use and the retention of a portion of the assembly for subsequent use.

A still further object of the present invention is to provide for a novel suppository insertion assembly in which the disposable portion of the assembly can be sterilly packaged.

A still further object of the present invention is to provide for a novel suppository insertion assembly in which the non-disposable portion of the assembly can be sterilized.

### SUMMARY OF THE INVENTION

A suppository insertion assembly having a first end having a sealable reservoir member and associated plunger and piston, the reservoir member in first conduit communication with a disposable suppository retaining member, the suppository retaining member having a second conduit means snap fit and securable with the first conduit means of the reservoir member, the reservoir member for receipt of a petroleum jelly cartridge, or similar pressurized fluid, the plunger-piston member of the reservoir member compressing the petroleum jelly cartridge so as to cause petroleum jelly to pass through the first and second conduit means and to exert pressure on the suppository, thereby ejecting the suppository from the suppository retention member into the rectum.

### BRIEF DESCRIPTION OF THE DRAWINGS

These and other objects of the present invention will become apparent, particularly when taken in light of the following illustrations wherein:

FIG. 1 is a side view of the suppository insertion assembly of the present invention;

FIG. 2 is a cross sectional view of the reservoir, plunger and piston members of the present invention;

FIG. 3 is a close up of the suppository retention member of the present invention; and

FIG. 4 is a schematic of a second embodiment of the suppository insertion assembly.

### DETAILED DESCRIPTION OF THE INVENTION

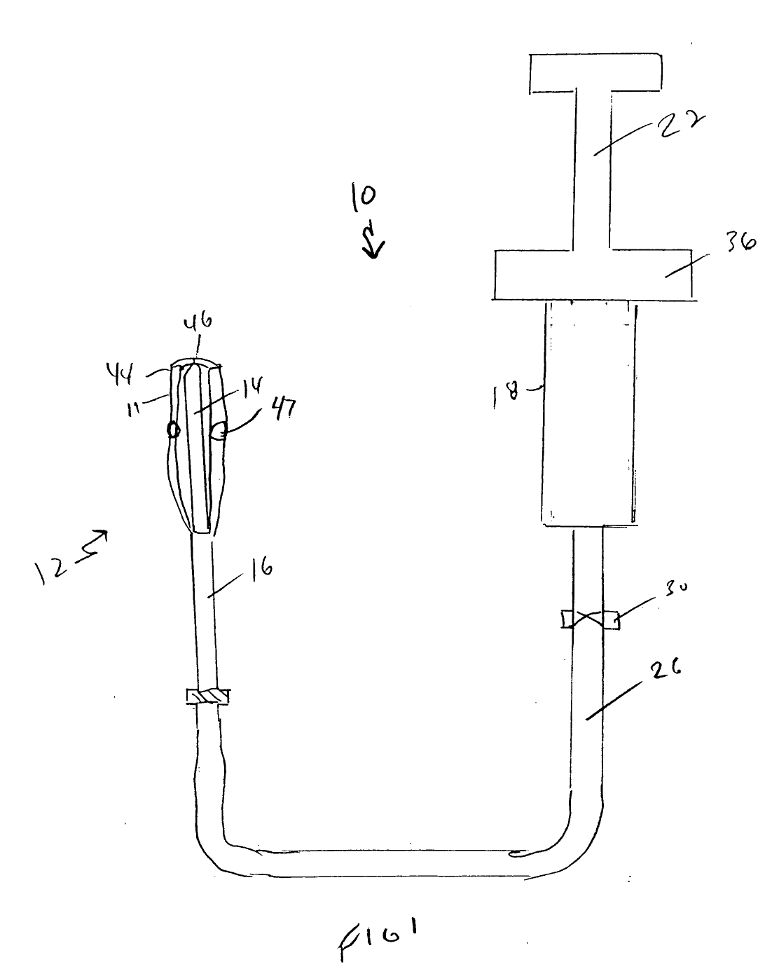

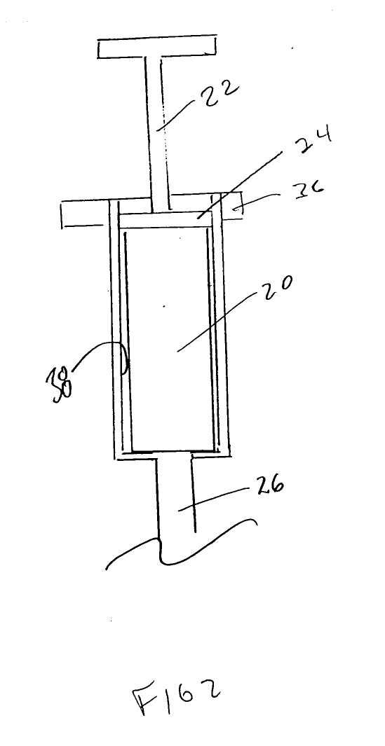

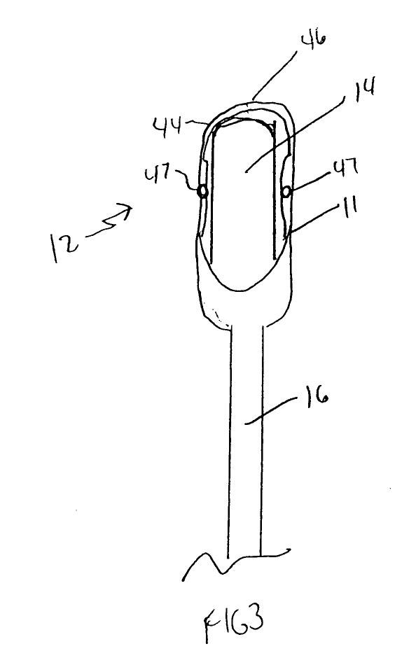

FIG. 1 is a side view of a suppository insertion assembly of the present invention, FIG. 2 is a cross sectional view of the reservoir, plunger and piston members of the present invention and FIG. 3 is a close up of the suppository retention member of the present invention.

The suppository insertion assembly 10 comprises a disposable suppository retention member 12, in the form of a housing 11 having a suppository 14 positioned therein, having a conduit portion 16 and a reservoir member 18 for receipt of a petroleum jelly cartridge or similar fluid cartridge 20, having associated therewith a plunger 22 and piston member 24. Reservoir member 18 further has a first conduit member 26 which is removable securable to a second conduit member 16 associated with the suppository retention member 12. Also positioned in first conduit member 26 from reservoir 18 is a vacuum relief valve 30.

The length of first conduit member 26 disposed from a reservoir member 18 is variable. The plunger 22 and piston member 24 are removable secured to reservoir member 18 by a screw or securable cap 36 which allows access to the interior cavity 38 of reservoir member 18. The first conduit member 26 emanating from reservoir member 18 may be flexible to allow for the individual to insert the suppository without assistance. Conduit member 16 may be configured in a straight section, which in turn would allow medical personnel to insert the suppository. The cavity 38 within reservoir member 18 is dimensioned to receive a preformed petroleum jelly or similar fluid cartridge 20 as illustrated in FIG. 2. The preformed petroleum jelly cartridge 20 would be sold in a sterile package with suppository retention member 12 and suppository 14.

The suppository retention member 12 encapsulates a suppository 14, the upper portion 44 of the suppository retention member 12 being formed with a membrane 46 which is penetratable by the suppository 14 under pressure as more fully described hereafter. This membrane 46 may be formed of any suitable material and may be formed with prestressed embossing lines to aid in its rupture to allow passage there through of the actual suppository 14, membrane 46 being secured by O-ring 47.

In operation, the user would purchase a sterilized package containing the suppository retention member 12, with suppository 14, and petroleum jelly cartridge 20. The suppository retention member 12 and suppository 14 would be secured to first conduit member 26 by means of second conduit member 16 extending from the suppository retention member 12 in either a threaded or push on connection. The relief valve 30 would be open in order to bleed air into the first conduit member 26. The cap member 36, plunger member 22, and piston member 24 could be removed from reservoir member 18. The petroleum jelly cartridge member 20 would then be slidably inserted into the cavity 38 of reservoir member 18. Cap member 36, piston member 24, and plunger member 22 would then be resecured to the reservoir member 18 with the plunger 22 and piston member 24 being in an extended position upwardly due to the presence of the petroleum jelly cartridge 20 within the reservoir. The relief valve 30 would then be closed. The suppository retention member 12 would then be inserted into the rectum of the individual either by the individual himself with the first conduit member 26 being flexible and resilient, or by a medical personnel individual with either a flexible conduit member 26 or a straight rigid conduit. The plunger member 22 is gradually depressed causing piston member 24 to exert pressure onto the petroleum jelly cartridge 20 forcing it through first conduit 26 of reservoir member 18 and through second conduit 16 of suppository retention member 12 exerting pressure upon suppository 14 within suppository retention member 12 thus forcing suppository 14 against membrane 46, causing membrane 46 to rupture and allow the suppository 14 to exit the suppository retention member and enter into the digestive tract.

Upon completion of the procedure, the suppository retention member 12 would be withdrawn and disconnected from first conduit member 26 extending from reservoir member 18 and the suppository retention member 12 would be discarded or disposed.

The reservoir member 18, and associated plunger 22, cap member 26 and piston member 24 and associated first conduit member 26 could also be made of disposable material for one time use, or made of sterilizable material for multi applications, since no portion of the reservoir member 18, plunger 22, piston member 24, or associated first conduit member therewith 26, comes into contact with the individual. Still further, if greater length of conduit 26 was desired, it could be mated to an additional length of similar conduit by means of an adjustable slip joint and O-ring seals.

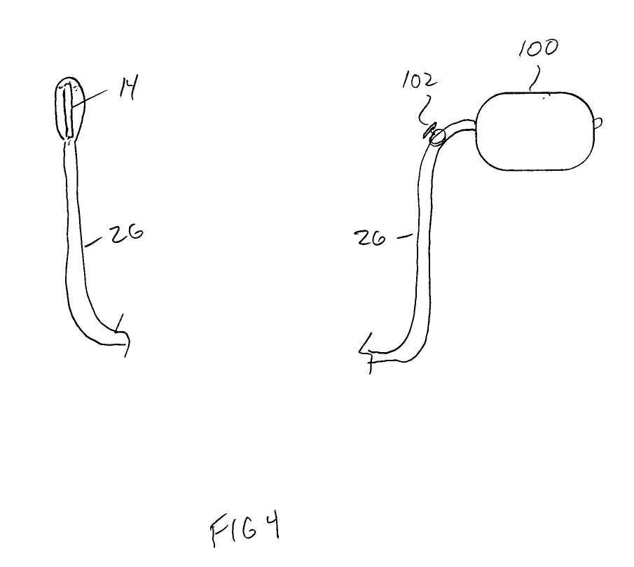

FIG. 4 is a second embodiment of the suppository insertion assembly of the present invention illustrating a second means of propelling the insertion package by means of pressurized air which could be supplied by means of a small hand held air pump 100 similar to that used on blood pressure testing equipment. A valve means 62 would allow pressure to be built up within the conduit 26 which in turn would exert a pressure upon the suppository 14 to effectuate its insertion.

While the present invention has been described with respect to the exemplary embodiments thereof, it will be recognized by those of ordinary skill in the art that many modifications or changes can be achieved without departing from the spirit and scope of the invention. Therefore it is manifestly intended that the invention be limited only by the scope of the claims and the equivalence thereof.

I claim:

1. A suppository insertion assembly for use by the individual or with an aide, the suppository insertion assembly comprising:
a disposable suppository retention member defined by a housing having an open lower end and a membrane upper end member, the disposable suppository retention member having a suppository slidably receivable therein through said lower open end and retained in position by said membrane upper end member, said disposable suppository retention member having a flexible conduit member removably securable to said lower end, said flexible conduit member in communication with a reservoir member, said flexible conduit member having a length sufficient to allow an individual user to insert said disposable suppository retention member and to operate said reservoir member, said reservoir member having a pressurized member for exerting pressure through said flexible conduit member to said disposable suppository retention member imposing force on said suppository sufficient to cause said suppository to rupture said membrane upper end member and penetrate the rectum.

2. The suppository insertion assembly in accordance with claim 1 wherein said reservoir member is defined by a housing member having a slidable piston member and plunger member removably secured to the upper end of said housing member, said lower end of said reservoir member being removably secured to said flexible conduit member, said reservoir member dimensioned to receive a fluid cartridge, said fluid cartridge having a fluid disposable outwardly from said reservoir member through said flexible conduit member to said disposable suppository retention member imposing force on said suppository sufficient to cause said suppository to rupture said membrane upper end member and penetrate the rectum.

3. The suppository insertion assembly in accordance with claim 1 wherein said flexible conduit member is formed with a bleed off relief valve between said disposable suppository retention member and said reservoir member to bleed said conduit member of air.

4. The suppository insertion assembly in accordance with claim 1 wherein said disposable suppository retention member is preformed with said suppository positioned therein.

5. The suppository insertion assembly in accordance with claim 1 wherein said disposable suppository retention member is designed to receive a separate suppository through its lower open end.

6. The suppository insertion assembly in accordance with claim 1 wherein said flexible conduit member, said reservoir member, said piston member, and said plunger member are disengagable to allow sterilization, reassembly and reuse.

7. The suppository insertion assembly in accordance with claim 1 wherein said flexible conduit member is of sufficient length and said fluid cartridge is of sufficient volume to allow an individual user to utilize the suppository insertion assembly to displace said suppository for the purpose intended without any aid.

8. A suppository insertion assembly in accordance with claim 1 wherein said reservoir member is defined by a housing member having a flexible pressurized member for exerting air pressure through said flexible conduit member to said disposable suppository retention member imposing a force of air on said suppository sufficient to cause said suppository to rupture said membrane upper end member and penetrate the rectum.
